SEQUENCE LISTING (16 sequences)

SEQ ID NO: 1 - DNA, 2756 bp, Homo sapiens
tcgagcggccgcccgggcaggtgtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagaaggaatctcaaaagaatgataaagatttgaagatattagagaaagagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggacaatattgttatattatctaaacaagtagaagatctaaatgtgaaatgtcagctgcttgaaacagaaaaagaagaccatgtcaacaggaatagagaacacaacgaaaatctaaatgcagagatgcaaaacttagaacagaagtttattcttgaacaacgggaacatgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggcttttctagtctgagcttctttagctaggctaaaacaccttggcttgttattgcctctactttgattcttgataatgctcacttggtcctacctattatcctttctacttgtccagttcaaataagaaataaggacaagcctaacttcatagtaacctctctatttt

SEQ ID NO: 2 - DNA, 2708 bp, Homo sapiens
tcgagcggccgcccgggcaggtgtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggacaatattgttatattatctaaacaagtagaagatctaaatgtgaaatgtcagctgcttgaaacagaaaaagaagaccatgtcaacaggaatagagaacacaacgaaaatctaaatgcagagatgcaaaacttagaacagaagtttattcttgaacaacgggaacatgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggcttttctagtctgagcttctttagctaggctaaaacaccttggcttgttattgcctctactttgattcttgataatgctcacttggtcctacctattatcctttctacttgtccagttcaaataagaaataaggacaagcctaacttcatagtaacctctctatttt

SEQ ID NO: 3 - DNA, 2609 bp, Homo sapiens
tcgagcggccgcccgggcaggtgtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagaaggaatctcaaaagaatgataaagatttgaagatattagagaaagagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggacaatattgttatattatctaaacaagtagaagatctaaatgtgaaatgtcagctgcttgaaacagaaaaagaagaccatgtcaacaggaatagagaacacaacgaaaatctaaatgcagagatgcaaaacttagaacagaagtttattcttgaacaacgggaacatgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggcttttctagtctgagcttctttagctaggctaaaacaccttggcttgttattgcctctactttgattcttgataatgctcacttggtcctacctattatcctttctacttgtccagttcaaataagaaataaggacaagcctaacttcatagtaacctctctatttt

SEQ ID NO: 4 - DNA, 22 bp, Homo sapiens
caggtcacccaaaggagtctcg

SEQ ID NO: 5 - DNA, 20 bp, Homo sapiens
caagctcatccagtgtttgc

SEQ ID NO: 6 - DNA, 23 bp, Homo sapiens
ggccgtcaacaagtcctttccta

SEQ ID NO: 7 - DNA, 24 bp, Homo sapiens
ttgggctattttcccttgagactc

SEQ ID NO: 8 - DNA, 20 bp, Homo sapiens
aggaggaacaagctgaaagg

SEQ ID NO: 9 - DNA, 20 bp, Homo sapiens
ttcctgagctgcaccatgtt

SEQ ID NO: 10 - DNA, 18 bp, Homo sapiens
gcatcgtgatggactccg

SEQ ID NO: 11 - DNA, 19 bp, Homo sapiens
gctggaaggtggacagcga

SEQ ID NO: 12 - DNA, 20 bp, Homo sapiens
cacgaaccacggcactgatt

SEQ ID NO: 13 - DNA, 22 bp, Homo sapiens
ttttcttgctgccagtctggac

SEQ ID NO: 14 - protein, 725 aa, Homo sapiens [sequence omitted]

SEQ ID NO: 15 - protein, 709 aa, Homo sapiens
MSFPKAPLKRFNDPSGCAPSPGAYDVKTLEVLKGPVSFQKSQRFKQQKESKQNLNVDKDTTLPASARKVKSSESKIRVLLQERGAQDRRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEDNIVILSKQVEDLNVKCQLLETEKEDHVNRNREHNENLNAEMQNLEQKFILEQREHEKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 16 - protein, 676 aa, Homo sapiens
MSFPKAPLKRFNDPSGCAPSPGAYDVKTLEVLKGPVSFQKSQRFKQQKESKQNLNVDKDTTLPASARKVKSSESKKESQKNDKDLKILEKEIRVLLQERGAQDRRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEDNIVILSKQVEDLNVKCQLLETEKEDHVNRNREHNENLNAEMQNLEQKFILEQREHEKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFERMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

(19) United States
(12) Patent Application Publication
(10) Pub. No.: US 2003/0170755 A1
(43) Pub. Date: Sep. 11, 2003

(54) Tumor-associated antigen RHAMM

(75) Inventors: Michael Schmitt, Ulm (DE); Jochen Greiner, Ulm (DE)

Correspondence Address: WOLF GREENFIELD & SACKS, PC, FEDERAL RESERVE PLAZA, 600 ATLANTIC AVENUE, BOSTON, MA, 02210-2211, US

(73) Assignee: Ludwig Institute for Cancer Research, New York, NY

(21) Appl. No.: 10/256,250

(22) Filed: Sep. 26, 2002

Related U.S. Application Data
(60) Provisional application No. 60/324,989, filed on Sep. 26, 2001.

(51) Int. Cl. G01N 33/574; C12M 1/34

(52) U.S. Cl. 435/7.23; 435/287.2

(57) ABSTRACT

The invention provides methods for diagnosing cancer including acute myeloid leukemia and chronic myeloid leukemia, based on the identification of certain cancer-associated polypeptides as antigens that elicit immune responses in cancer. The identified antigens can be utilized as markers for diagnosing cancer, and for following the course of treatment of cancer.

### RELATED APPLICATIONS

This application claims priority under 35 U.S.C. §119 from U.S. provisional application serial No. 60/324,989, filed Sep. 26, 2001.

### FIELD OF THE INVENTION

The invention relates to use of novel tumor-associated antigens in the diagnosis of cancer, including acute and chronic myeloid leukemia.

### BACKGROUND OF THE INVENTION

The myeloid leukemias are members of a heterogeneous group of diseases characterized by infiltration of the blood, bone marrow, and other tissues by neoplastic cells of the hematopoietic system. There is a spectrum of symptoms of the mycloid leukemias, which range from to slowly progressive to rapidly fatal. (see; Harrison's Principles of Internal Medicine, 14/e, McGraw-Hill Companies, New York, 1998). Myeloid leukemias are categorized as either acute myeloid leukemia (AML) or chronic myeloid leukemia (CML) and they differ in their progression and prognosis. The onset of AML may be genetically based, or the result of exposure to radiation, chemicals, or drugs such as antineoplastic drugs used in cancer treatment. CML is also linked to chromosomal abnormalities and its progression is influenced by exposure to radiation and/or chemicals.

Treatment of patients with acute myeloid leukemia has become more effective during the last three decades through the development of polychemotherapy and hematopoietic stem cell transplantation (HSCT). Today, the majority of the AML patients reach a morphologically complete remission (CR) (Phillips G L, et al., Blood 77:1429-1435, 1991). However, often a CR is not durable, and a high percentage of these patients relapse (Cassileth P A, et al., Blood 79:1924-1930 1992). Allogeneic hematopoietic stem cell transplantation (HSCT) is a therapeutic option (Zittoun R A, et al., N Engl J Med 332:217-223, 1995) based on the effects of irradiation and high-dose chemotherapy for these patients, and also includes induction of the graft versus leukemia (GVL) effect (Kolb H J, et al., Blood 76:2462-2465 1990). Treatment options for CML parallel those for AML.

An important factor in the success of treatment for AML and CML is the length of time between the onset of the disease and the start of treatment. Appropriate methods for diagnosis are important for rapid, accurate identification of these disorders. A second factor in the survival time of patients treated for AML or CML, is the use of methods to monitor the status of the disease during and after treatment. The ability to monitor the progression or regression of the disease enables health care providers to respond rapidly to the therapeutic needs of the patients and thereby augment the probability of a positive outcome for the patient.

### SUMMARY OF THE INVENTION

The invention provides methods for diagnosing myeloid leukemias based on the identification of certain RHAMM polypeptides and the nucleic acid molecules that encode the polypeptides. The leukemia-associated polypeptides of the invention are RHAMM polypeptides and these polypeptides or fragments thereof are antigens that elicit immune responses in myeloid leukemias. The identified antigens and/or the nucleic acid molecules that encode them can be utilized as markers for diagnosing myeloid leukemias, for following the course of treatment of myeloid leukemias, and for assessing myeloid leukemia remission and treatments.

According to one aspect of the invention, methods for diagnosing cancer in a subject are provided. The methods involve obtaining a biological sample from a subject, contacting the sample with a RHAMM polypeptide encoded by a nucleic acid molecule comprising a nucleotide sequence selected from the group consisting of SEQ ID NO: 1, 2, and 3, and determining specific binding between the RHAMM polypeptide and agents in the sample, wherein the presence of specific binding is diagnostic for cancer in the subject.

According to another aspect of the invention, methods for determining onset, progression, or regression, of cancer in a subject are provided. The methods involve: obtaining from a subject at a first time a first biological sample, determining specific binding between agents in the first sample and a RHAMM polypeptide encoded by a nucleic acid molecule comprising a nucleotide sequence selected from the group consisting of SEQ ID NO: 1, 2, and 3. The method further involves obtaining from a subject at a subsequent time a second biological sample, determining specific binding between agents in the second sample and a RHAMM polypeptide encoded by a nucleic acid molecule comprising a nucleotide sequence selected from the group consisting of SEQ ID NO: 1, 2, and 3, and comparing the determination of binding in the first sample to the determination of specific binding in the second sample as a determination of the onset, progression, or regression of the cancer.

According to yet another aspect of the invention, methods for selecting a course of treatment of a subject having or suspected of having cancer are provided. The methods involve obtaining from the subject a biological sample, contacting the sample with a RHAMM polypeptide encoded by a nucleic acid molecule comprising a nucleotide sequence selected from the group consisting of SEQ ID NO: 1, 2, and 3, and determining specific binding between agents in the sample that are differentially expressed in different types of cancer, and the RHAMM polypeptide, and selecting a course of treatment appropriate to the cancer of the subject. In some embodiments, the treatment is administering antibodies that specifically bind to the RHAMM polypeptide. In certain embodiments, the antibodies are labeled with one or more cytotoxic agents.

In some embodiments of the foregoing methods, the sample is blood. In certain embodiments of the foregoing methods, the agents are antibodies or antigen-binding fragments thereof. In some embodiments of the foregoing methods, the cancer is acute or chronic myeloid leukemia. In some embodiments of the foregoing methods, the cancer is selected from the group consisting of: renal cell cancer, metastatic melanoma, and ovarian carcinoma.

According to another aspect of the invention, kits for the diagnosis of cancer in a subject are provided. The kits include a RHAMM polypeptide encoded by a nucleic acid molecule comprising a nucleotide sequence selected from the group consisting of SEQ ID NO: 1, 2, and 3, and one or more control antigens; and instructions for the use of the polypeptide and control antigens in the diagnosis of cancer. In some embodiments, the RHAMM polypeptide is bound to a substrate. In certain embodiments, the one or more agents are antibodies or antigen-binding fragments thereof. In some embodiments, the cancer is acute or chronic myeloid leukemia. In some embodiments, the cancer is selected from the group consisting of: renal cell cancer, metastatic melanoma, and ovarian carcinoma.

According to yet another aspect of the invention, protein microarrays are provided. The protein microarrays include a RHAMM polypeptide, wherein the RHAMM polypeptide is encoded by a nucleic acid molecule comprising a nucleotide sequence selected from the group consisting of SEQ ID NO: 1, 2, and 3, and fixed to a solid substrate. In some embodiments, the protein microarray also includes at least one control polypeptide molecule.

### BRIEF DESCRIPTIONS OF THE DRAWINGS

The figures are illustrative only and are not required for enablement of the invention disclosed herein.

FIG. 1 is a digitized image showing RHAMM mRNA expression (PCR 2) analysis of different normal tissues using a normalized cDNA tissue panel (Multiple tissue cDNA panels, BK 1420-1 and BK 1420-2, Clontech, Palo, Calif., USA). High mRNA expression was only found in normal testis, placenta and thymus tissue. Very low mRNA expression was detectable in pancreas and lung tissue, but in none of the other tissues (FIGS. 1A, B). β-actin was used as a housekeeping gene (FIGS. 1C, D).

FIG. 2 is a digitized image showing mRNA expression (PCR 1) of the antigen RHAMM examined during the course of induction polychemotherapy (idarubicine, arabinoside, etoposide) in an AML patient. High mRNA expression of RHAMM was detectable in the untreated AML patient with high counts of blasts in the bone marrow (BM, lane 1), and low mRNA expression in PBMN (lane 2). The therapy induced a complete remission of the leukemia after two courses. In the state of complete remission, no mRNA expression of RHAMM was detectable even in the BM (lane 3). K562 served as a positive control. β-actin was expressed at an equal level in all samples.

FIG. 3 is a digitized image illustrating Western blot analysis of the antigen RHAMM performed in leukemia cell lines, AML patients, and PBMN of healthy volunteers using a rabbit polyclonal antibody specific for the human 85-90 kDa RHAMM protein. The leukemia cell line K562 was used as a positive control (lane 1). One patient was suffering from a diffuse rapid progressive anaplastic carcinoma (lane 5). The figure shows high protein expression of the leukemia cell line K562 and the other leukemia cell lines HL-60 (lane 2), Kasumi-1 (lane 3), KG-1, and Oci-5 (lane 4). Protein expression was detectable in the AML patient (lane 7), whereas no expression was found in PBMN of healthy volunteers (lanes 6 and 8).

### DESCRIPTION OF THE SEQUENCES

| | | |
|---|---|---|
| RHAMM^{FL} (Full Length) nucleotide sequence | | SEQ ID NO:1 |
| RHAMM⁻⁴⁸ nucleotide sequence | | SEQ ID NO:2 |
| RHAMM⁻¹⁴⁷ nucleotide sequence | | SEQ ID NO:3 |
| RHAMM PCR1, forward primer: | 5′ CAGGTCACCCAAAGGAGTCTCG 3′ | SEQ ID NO:4 |
| RHAMM PCR1 reverse primer: | 5′ CAAGCTCATCCAGTGTTTGC 3′ | SEQ ID NO:5 |
| RHAMM PCR2 forward primer: | 5′ GGCCGTCAACAAGTCCTTTCCTA 3′. | SEQ ID NO:6 |
| RHAMM PCR2 reverse primer: | 5′ TTGGGCTATTTTCCCTTGAGACTC 3′. | SEQ ID NO:7 |
| RHAMM PCR3 forward primer: | 5′ AGGAGGAACAAGCTGAAAGG 3′. | SEQ ID NO:8 |
| RHAMM PCR3 reverse primer: | 5′ TTCCTGAGCTGCACCATGTT 3′. | SEQ ID NO:9 |
| β-Actin forward primer: | 5′ GCATCGTGATGGACTCCG 3′. | SEQ ID NO:10 |
| β-Actin reverse primer: | 5′ GCTGGAAGGTGGACAGCGA 3′. | SEQ ID NO:11 |
| TBP: forward primer: | 5′ CACGAACCACGGCACTGATT 3′ | SEQ ID NO:12 |
| TBP: reverse primer: | 5′ TTTTCTTGCTGCCAGTCTGGAC 3′. | SEQ ID NO:13 |
| RHAMM^{FL} (Full Length) amino acid sequence | | SEQ ID NO:14 |
| RHAMM⁻⁴⁸ acid sequence | | SEQ ID NO:15 |
| RHAMM⁻¹⁴⁷ amino acid sequence | | SEQ ID NO:16 |

### DETAILED DESCRIPTION

The invention described herein relates to the identification of polypeptides that elicit specific immune responses in subjects with cancer, particularly myeloid leukemias such as AML and CML. Myeloid leukemia-associated polypeptides (SEQ ID NO: 14-16) are RHAMM polypeptides, which have been identified through SEREX screening of patients with myeloid leukemia. The SEREX method (serological analysis of antigens by recombinant expression cloning), has been described by Sahin et al. (Proc. Natl. Acad. Sci. USA 92:11810-11813, 1995). The RHAMM polypeptides and the encoding nucleic acid molecules thereof may be used as markers for myeloid leukemias, including AML and CML, and may be used in the diagnosis and treatment assessment of AML and CML in humans. The markers may also be utilized to diagnose and select and monitor treatment of other cancers, including, but not limited to metastatic melanoma, renal cell cancer, breast cancer, and ovarian cancer.

Polypeptides that elicit specific immune responses in myeloid leukemias have now been identified and this identification allows use of these RHAMM polypeptides or the encoding nucleic acids molecules thereof in myeloid leukemia diagnostic assays and kits. Such assays and kits are useful to detect myeloid leukemia in human subjects, and for staging the progression, regression, or onset of myeloid leukemia in subjects. The methods and kits described herein may also be used to evaluate treatments for myeloid leukemias.

In addition to the above-described uses of the invention, the invention may also be used in the diagnosis and treatment assessment of various other cancers including, but not limited to: renal cell cancer, metastatic melanoma, breast cancer, and ovarian cancer.

As used herein, the RHAMM polypeptides are polypeptides encoded by an isolated nucleic acid molecule (SEQ ID 1, 2, or 3) selected from the group consisting of: (a) nucleic acid molecules that hybridize under highly stringent conditions to a nucleic acid molecule having a nucleotide sequence set forth as SEQ ID NO: 1, 2, or 3 and which codes for a RHAMM protein, (b) nucleic acid molecules that differ from the nucleic acid molecules of (a) in codon sequence due to the degeneracy of the genetic code, and (c) complements of (a) or (b).

As used herein, “myeloid leukemia-associated polypeptides” means RHAMM polypeptides that elicit specific immune responses in animals having myeloid leukemia and thus include RHAMM antigens and fragments of RHAMM antigens, that are recognized by the immune system (e.g., by antibodies and/or T lymphocytes). The invention also relates to the use of the nucleic acid molecules that encode the RHAMM polypeptides. In all embodiments, human RHAMM polypeptides and the encoding nucleic acid molecules thereof, are preferred. As used herein, the “encoding nucleic acid molecules thereof” means the nucleic acid molecules that code for the polypeptides.

As used herein, a subject is preferably a human, non-human primate, cow, horse, pig, sheep, goat, dog, cat, or rodent. In all embodiments, human subjects are preferred. In some embodiments the subject is suspected of having cancer, and in preferred embodiments the subject is suspected of having myeloid leukemia. In some embodiments the subject has been diagnosed with cancer, and in preferred embodiments the subject has been diagnosed with myeloid leukemia.

As used herein, “different types” of cancer may include but are not limited to: breast cancer, melanoma, ovarian cancer, renal cell cancer, and myeloid leukemia, which includes AML and CML. In addition, as used herein “different types” of cancer may mean different cell types, and different stages of cancer (e.g., primary tumor or metastatic growth).

Methods for identifying subjects suspected of having mycloid leukemia may include but are not limited to: patient physical examination, subject's medical history, blood chemistries (e.g. for serum electrolytes, uric acid, creatinine, hepatic enzymes, etc.), assays for hemoglobin level, leukocyte count (differential), platelet count, bone marrow biopsy, and lumbar puncture. Traditional diagnostic methods for myeloid leukemia and the clinical delineation of myeloid leukemia diagnoses are well known to those of skill in the medical arts.

As used herein, a biological sample includes, but is not limited to: tissue, cells, or body fluid (e.g. blood, lymph node fluid, or spinal fluid). The fluid sample may include cells and/or fluid. The tissue and cells may be obtained from a subject or may be grown in culture (e.g. from a cell line).

As used herein, a biological sample is tissue or cells obtained using methods well known to those of ordinary skill in the related medical arts (e.g., from blood drawing, tissue biopsy, or aspiration). The phrase “suspected of being cancerous” as used herein means a myeloid leukemia tissue sample suspected by one of ordinary skill in the medical arts to contain cancerous cells.

In preferred embodiments, the RHAMM nucleic acid molecules include molecules selected from the group of nucleotide sequences numbered 1 through 3 in Table 3 (SEQ ID Nos: 1-3) and the RHAMM polypeptides are selected from the group of polypeptide sequences encoded by SEQ ID NO: 1-3, (SEQ ID NOs: 14-16) in Table 3. In some embodiments, RHAMM nucleotides may include nucleotides other than those comprising a nucleotide sequence selected from the group consisting of SEQ ID NO: 1-3. In addition, in some embodiments, RHAMM polypeptides may include polypeptides other than those encoded by nucleic acid molecules comprising a nucleotide sequence selected from the group consisting of SEQ ID NOs: 1-3.

The invention involves in some embodiments, diagnosing, or monitoring cancer, such as myeloid leukemia, in subjects by determining the presence of an immune response to one or more of the RHAMM polypeptides described herein. In preferred embodiments, this determination is performed by assaying a bodily fluid obtained from the subject, preferably blood, lymph node fluid, or spinal fluid, for the presence of antibodies against one or more RHAMM polypeptides or the nucleic acid molecules that encode the RHAMM polypeptides.

Measurement of the immune response against one of the RHAMM polypeptides described herein, in a subject over time by sequential determinations permits monitoring of the disease and/or the effects of a course of treatment. For example, a sample, such as blood or spinal fluid, may be obtained from a subject, tested for an immune response to one of the RHAMM polypeptides, and at a second, subsequent time, another sample, such as blood or spinal fluid, may be obtained from the subject and similarly tested. The results of the first and second (subsequent) tests can be compared as a measure of the onset, regression, or progression of myeloid leukemia, or, if myeloid leukemia treatment was undertaken during the interval between obtaining the samples, the effectiveness of the treatment may be evaluated by comparing the results of the two tests.

In all embodiments, treatment for myeloid leukemia may include, but is not limited to: hematopoietic stem cell transplantation, allogeneic transplantation, donor lymphocyte infusion, and remission induction chemotherapy, e.g. polychemotherapy.

In a preferred embodiment, treatment may include administering antibodies that specifically bind to the RHAMM antigen. Optionally, an antibody can be linked to one or more detectable markers, antitumor agents or immunomodulators. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope may be an alpha-emitting isotope such as ²²⁵Ac, ²¹¹At, ²¹²Bi, or ²¹³Bi. Alternatively, the cytotoxic radionuclide may be a beta-emitting isotope such as ¹⁸⁶Rh, ¹⁸⁸Rh, ⁹⁰Y, ¹³¹I or ⁶⁷Cu. Further, the cytotoxic radionuclide may emit Auger and low energy electrons such as the isotopes ¹²⁵I, ¹²³I or ⁷⁷Br.

Suitable chemical toxins or chemotherapeutic agents include members of the enediyne family of molecules, such as chalicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouaracil. Other chemotherapeutic agents are known to those skilled in the art.

Agents that act on the tumor neovasculature can include tubulin-binding agents such as combrestatin A4 (Griggs et al., Lancet Oncol. 2:82, 2001) and angiostatin and endostatin (reviewed in Rosen, Oncologist 5:20, 2000, incorporated by reference herein). Immunomodulators may also be conjugated to RHAMM antibodies.

The invention thus involves in one aspect, the association with myeloid leukemia of polypeptides, genes encoding those polypeptides, functional modifications and variants of the foregoing, useful fragments of the foregoing, as well as diagnostics relating thereto, and diagnostic uses thereof. In some embodiments, the RHAMM polypeptide genes correspond to SEQ ID NOs: 1-3. Encoded polypeptides (e.g., proteins), peptides and antisera thereto are also preferred for diagnosis and correspond to SEQ ID NOs: 14-16. In some embodiments, the method or kit may include RHAMM polypeptide genes in addition to those corresponding to SEQ ID NOs. 1-3 and the encoded polypeptides (e.g. proteins), peptides, and antisera thereto in addition to those corresponding to SEQ ID NOs: 14-16.

The amino acid sequences identified by SEREX as myeloid leukemia-associated polypeptides, and the nucleotide sequences encoding them, are sequences deposited in databases such as GenBank. The use of the identified sequences in diagnostic assays for myeloid leukemia is novel, as is the use of one or more of the sequences in kits (e.g. diagnostic kits).

Homologs and alleles of the RHAMM polypeptide encoding nucleic acids of the invention can be identified by conventional techniques. Thus, an aspect of the invention is those nucleic acid sequences that code for RHAMM antigens and antigenic fragments thereof. As used herein, a homolog of a RHAMM polypeptide is a polypeptide from a human or other animal that has a high degree of structural similarity to the identified RHAMM polypeptides.

Identification of human and other organism homologs of RHAMM polypeptides will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., peripheral blood mononuclear cells [PBMN]) and use the nucleic acids that encode the RHAMM polypeptides identified herein to screen the library for related nucleotide sequences. The screening preferably is performed using high-stringency conditions to identify those sequences that are closely related by sequence identity. Nucleic acids so identified can be translated into polypeptides and the polypeptides can be tested for activity.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of RHAMM polypeptide encoding nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

In general, homologs and alleles typically will share at least 90% nucleotide identity and/or at least 95% amino acid identity to the sequences of RHAMM antigens, antigenic fragments, and antigen precursors of polypeptides and the nucleic acids that encode them respectively. In some instances the nucleic acids and/or polypeptides will share at least 95% nucleotide identity and/or at least 97% amino acid identity, and in other instances will share at least 97% nucleotide identity and/or at least 99% amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for RHAMM polypeptide genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of RHAMM polypeptide nucleic acids, Northern blot hybridizations using the foregoing conditions can be performed on samples taken from myeloid leukemia patients or subjects suspected of having a condition characterized by abnormal cell proliferation or hematological malignancies. Amplification protocols such as polymerase chain reaction using primers that hybridize to the sequences presented also can be used for detection of the RHAMM polypeptide genes or expression thereof.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., bone marrow). One also can use expression cloning utilizing the antisera described herein to identify nucleic acids that encode related antigenic proteins in humans or other species using the SEREX procedure to screen the appropriate expression libraries. (See: Sahin et al. Proc. Natl. Acad. Sci. USA 92:11810-11813, 1995).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating myeloid leukemia-associated polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1, 2, 3, 4, 5, 6, 7, 8, 9, 10, 11, 2, 13, 14, 15, 16, 17, 18, 19, or 20 nucleotides). In preferred embodiments, these modified nucleic acid molecules and/or the polypeptides they encode retain at least one activity or function of the unmodified nucleic acid molecule and/or the polypeptides, such as antigenicity, receptor binding, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under highly stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides nucleic acid molecules that encode antigenic fragments of RHAMM proteins.

Fragments can be used as probes in Southern and Northern blot assays to identify such nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the RHAMM polypeptides, useful, for example, in the preparation of antibodies, and in immunoassays. Preferred fragments are antigenic fragments, which are recognized by agents that specifically bind to RHAMM polypeptides. As used herein, RHAMM antibodies are antibodies that specifically bind to RHAMM polypeptides.

The invention also permits the construction of RHAMM polypeptide gene “knock-outs” or “knock-ins” in cells and in animals, providing materials for studying certain aspects of myeloid leukemia and immune system responses to myeloid leukemia by regulating the expression of RHAMM polypeptides. For example, a knock-in mouse may be constructed and examined for clinical parallels between the model and a myeloid leukemia mouse with upregulated expression of a RHAMM polypeptide, which may be useful to trigger an immune reaction to the polypeptide. Such a cellular or animal model may also be useful for assessing treatment strategies for myeloid leukemia.

Alternative types of animal models for myeloid leukemia may be developed based on the invention. Stimulating an immune response to a RHAMM polypeptide in an animal may provide a model in which to test treatments, and assess the etiology of myeloid leukemia.

The invention also provides isolated polypeptides (including whole proteins and partial proteins) encoded by the foregoing RHAMM nucleic acids. Such polypeptides are useful, for example, alone or as fusion proteins to generate antibodies, and as components of an immunoassay or diagnostic assay. RHAMM polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, such as RHAMM antigen fragments including antigenic peptides also can be synthesized chemically using well-established methods of peptide synthesis.

Fragments of a polypeptide preferably are those fragments that retain a distinct functional capability of the polypeptide. Functional capabilities that can be retained in a fragment of a polypeptide include interaction with antibodies (e.g. antigenic fragments), interaction with other polypeptides or fragments thereof, selective binding of nucleic acids or proteins, and enzymatic activity. One important activity is the ability to provoke in a subject an immune response. As will be recognized by those skilled in the art, the size of the fragment will depend upon factors such as whether the epitope recognized by an antibody is a linear epitope or a conformational epitope. Thus, some antigenic fragments of RHAMM polypeptides will consist of longer segments while others will consist of shorter segments, (e.g. 5, 6, 7, 8, 9, 10, 11 or 12 or more amino acids long, including each integer up to the full length of the RHAMM polypeptide). Those skilled in the art are well versed in methods for selecting antigenic fragments of proteins.

The skilled artisan will also realize that conservative amino acid substitutions may be made in RHAMM polypeptides to provide functionally equivalent variants, or homologs of the foregoing polypeptides, i.e, the variants retain the functional capabilities of the RHAMM antigen polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the myeloid leukemia associated polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide is a RHAMM polypeptide, one can make conservative amino acid substitutions to the amino acid sequence of the peptide, and still have the polypeptide retain its specific antibody-binding characteristics.

Conservative amino-acid substitutions in the amino acid sequence of RHAMM polypeptides to produce functionally equivalent variants of RHAMM polypeptides typically are made by alteration of a nucleic acid encoding a RHAMM polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a RHAMM polypeptide. Where amino acid substitutions are made to a small unique fragment of a RHAMM polypeptide, such as an antigenic epitope recognized by autologous or allogeneic sera or cytolytic T lymphocytes, the substitutions can be made by directly synthesizing the peptide. The activity of functionally equivalent fragments of RHAMM polypeptides can be tested by cloning the gene encoding the altered RHAMM polypeptide into a bacterial or mammalian expression vector, introducing the vector into an appropriate host cell, expressing the altered polypeptide, and testing for a functional capability of the RHAMM polypeptides as disclosed herein. Peptides that are chemically synthesized can be tested directly for function, e.g., for binding to antisera recognizing associated antigens.

The isolation and identification of RHAMM polypeptides also permits the artisan to diagnose a disorder characterized by expression of RHAMM polypeptides, and characterized preferably by an immune response against the RHAMM polypeptides.

The methods related to RHAMM polypeptide immune responses involve determining the immune response (antibody or cellular) against one or more RHAMM polypeptides. The immune response can be assayed by any of the various immunoassay methodologies known to one of ordinary skill in the art. For example, the antigenic RHAMM polypeptides can be used as a target to capture antibodies from a sample, such as a blood sample drawn from a patient in an ELISA assay.

The methods related to RHAMM polypeptide expression involve determining expression of one or more RHAMM nucleic acids, and/or encoded RHAMM polypeptides and/or peptides derived therefrom and comparing the expression with that in a myeloid leukemia-free subject. Such determinations can be carried out via any standard nucleic acid determination assay, including the polymerase chain reaction, or assaying with labeled hybridization probes. Such hybridization methods include, but are not limited to microarray techniques.

The invention also makes it possible to isolate proteins that specifically bind to RHAMM antigens as disclosed herein, including antibodies and cellular binding partners of the RHAMM polypeptides. Additional uses are described further herein.

The invention also involves agents such as polypeptides that bind to RHAMM polypeptides. Such binding agents can be used, for example, in screening assays to detect the presence or absence of RHAMM polypeptides and complexes of RHAMM polypeptides and their binding partners and in purification protocols to isolate RHAMM polypeptides and complexes of RHAMM polypeptides and their binding partners. Such agents also may be used to inhibit the native activity of the RHAMM polypeptides, for example, by binding to such polypeptides.

The invention, therefore, embraces peptide binding agents which, for example, can be antibodies or fragments of antibodies having the ability to selectively bind to RHAMM polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to RHAMM polypeptides, and complexes of both RHAMM polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the RHAMM polypeptide. This process can be repeated through several cycles of reselection of phage that bind to the RHAMM polypeptide. Repeated rounds lead to enrichment of phage bearing particular sequences. DNA sequence analysis can be conducted to identify the sequences of the expressed polypeptides. The minimal linear portion of the sequence that binds to the RHAMM polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the RHAMM polypeptides.

Thus, the RHAMM polypeptides of the invention, including fragments thereof, can be used to screen peptide libraries, including phage display libraries, to identify and select peptide binding partners of the RHAMM polypeptides of the invention. Such molecules can be used, as described, for screening assays, for purification protocols, for interfering directly with the functioning of RHAMM polypeptides and for other purposes that will be apparent to those of ordinary skill in the art. For example, isolated RHAMM polypeptides can be attached to a substrate (e.g., chromatographic media, such as polystyrene beads, or a filter), and then a solution suspected of containing the binding partner may be applied to the substrate. If a binding partner that can interact with RHAMM polypeptides is present in the solution, then it will bind to the substrate-bound RHAMM polypeptide. The binding partner then may be isolated.

As detailed herein, the foregoing antibodies and other binding molecules may be used for example, to identify tissues expressing protein or to purify protein. Antibodies also may be coupled to specific diagnostic labeling agents for imaging of cells and tissues that express RHAMM polypeptides or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, and nuclides for nuclear magnetic resonance such as fluorine and gadolinium.

The invention also includes methods to monitor the onset, progression, or regression of myeloid leukemia or other cancers such as metastatic lymphoma, renal cell cancer, breast cancer or ovarian cancer in a subject by, for example, obtaining samples at sequential times from a subject and assaying such samples for the presence and/or absence of an antigenic response that is a marker of the condition. A subject may be suspected of having myeloid leukemia or may be believed not to have myeloid leukemia and in the latter case, the sample may serve as a normal baseline level for comparison with subsequent samples.

Onset of a condition is the initiation of the changes associated with the condition in a subject. Such changes may be evidenced by physiological symptoms, or may be clinically asymptomatic. For example, the onset of myeloid leukemia may be followed by a period during which there may be RHAMM physiological changes in the subject, even though clinical symptoms may not be evident at that time. The progression of a condition follows onset and is the advancement of the physiological characteristics of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

A marker for myeloid leukemia, or other cancer such as metastatic melanoma, breast cancer, renal cell cancer or ovarian cancer, may be the specific binding of a RHAMM polypeptide with an antibody. Onset of a myeloid leukemia, or other cancer condition may be indicated by the appearance of such a marker(s) in a subject's samples where there was no such marker(s) determined previously. For example, if marker(s) for myeloid leukemia are determined not to be present in a first sample from a subject, and myeloid leukemia marker(s) are determined to be present in a second or subsequent sample from the subject, it may indicate the onset of myeloid leukemia.

Progression and regression of a myeloid leukemia or other cancer condition may be generally indicated by the increase or decrease, respectively, of marker(s) in a subject's samples over time. For example, if marker(s) for myeloid leukemia are determined to be present in a first sample from a subject and additional marker(s) or more of the initial marker(s) for myeloid leukemia are determined to be present in a second or subsequent sample from the subject, it may indicate the progression of myeloid leukemia. Regression of myeloid leukemia may be indicated by finding that marker(s) determined to be present in a sample from a subject are not determined to be found, or found at lower amounts in a second or subsequent sample from the subject. For example, high levels of RHAMM mRNA expression AML patients have been shown to drop to undetectable levels following induction polychemotherapy resulting in a complete remission in peripheral blood and bone marrow. (see Example Section).

The progression and regression of a myeloid leukemia condition, or other cancer such as breast cancer, metastatic melanoma, ovarian cancer, or renal cell cancer, may also be indicated based on characteristics of the RHAMM polypeptides determined in the subject. For example, some RHAMM polypeptides may be abnormally expressed at specific stages of myeloid leukemia or other cancer (e.g. early-stage RHAMM polypeptides; mid-stage RHAMM polypeptides; and late-stage RHAMM polypeptides). Another example, although not intended to be limiting, is that RHAMM polypeptides may be differentially expressed in primary tumors versus metastases, thereby allowing the stage and/or diagnostic level of the disease to be established, based on the identification of selected RHAMM polypeptides in a subject sample.

Another method of staging myeloid leukemia may be based on variation in a subject's immune response to RHAMM polypeptides, which may or may not be abnormally expressed in the subject. Variability in the immune response to the polypeptides may be used to indicate the stage of myeloid leukemia (or other above-mentioned cancer) in a subject, for example, some RHAMM polypeptides may trigger an immune response at different stages of the myeloid leukemia or cancer than that triggered by other RHAMM polypeptides.

Different types of myeloid leukemia, including, but not limited to: AML and CML, may express different RHAMM polypeptides and the encoding nucleic acid molecules thereof, or may have different spatial or temporal expression patterns. Such variations may allow myeloid leukemia-specific diagnosis and subsequent treatment tailored to the patient's specific condition. These myeloid leukemia-specific diagnoses may also be based on the variations in immune responses to the different RHAMM polypeptides. Different type of cancers, including but not limited to: breast cancer, renal cell cancer, ovarian cancer, or melanoma, may also have different spatial or temporal expression patterns, which likewise may allow RHAMM polypeptide specific diagnosis and treatment tailored to an individual patient.

The invention includes kits for assaying the presence of RHAMM polypeptides and/or antibodies that specifically bind to RHAMM polypeptides. An example of such a kit may include the above-mentioned polypeptides bound to a substrate, for example a dipstick, which is dipped into a blood or body fluid sample of a subject. The surface of the substrate may then be processed using procedures well known to those of skill in the art, to assess whether specific binding occurred between the polypeptides and agents (e.g. antibodies) in the subject's sample. For example, procedures may include, but are not limited to, contact with a secondary antibody, or other method that indicates the presence of specific binding.

The foregoing kits can include instructions or other printed material on how to use the various components of the kits for diagnostic purposes.

The invention further includes nucleic acid or protein microarrays with RHAMM peptides or nucleic acids encoding such polypeptides. In this aspect of the invention, standard techniques of microarray technology are utilized to assess expression of the RHAMM polypeptides and/or identify biological constituents that bind such polypeptides. The constituents of biological samples include antibodies, lymphocytes (particularly T lymphocytes), and the like. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Nucleic acid arrays, particularly arrays that bind RHAMM peptides, also can be used for diagnostic applications, such as for identifying subjects that have a condition characterized by RHAMM polypeptide expression.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

Targets are peptides or proteins and may be natural or synthetic.

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, January 1999, the entire contents of which is incorporated by reference herein.

According to the present invention, nucleic acid microarray substrates may include but are not limited to glass, silica, aluminosilicates, borosilicates, metal oxides such as alumina and nickel oxide, various clays, nitrocellulose, or nylon. In all embodiments, a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

In some embodiments, one or more control nucleic acid molecules are attached to the substrate. Preferably, control nucleic acid molecules allow determination of factors such as binding characteristics, reagent quality and effectiveness, hybridization success, and analysis thresholds and success.

### EXAMPLES

Introduction

The expression of RHAMM was examined in leukemic blasts, solid tumor cells, and normal tissues. The association of RHAMM with metastasis described in breast cancer (Wang C, et al., Clin Can Res 4:567-576 (1998) was confirmed in melanoma. The expression pattern of RHAMM was evaluated by serological analysis, RT-PCR and western blot analysis, which demonstrated that RHAMM may serve as a target structure for a specific immunotherapy of leukemias and solid tumors.

Methods

Culture of Leukemia Cell Lines

Cell lines were cultured in RPMI 1640 (Biochrom, Berlin, Germany), 10% (v/v) fetal calf serum (FCS), 2 mM L-glutamine, 100 units/ml penicillin, and 100 units/ml streptomycin, using standard procedures. The cell lines were as follows: 1) K-562 (CML, blast crisis), 2) HL-60 (AML M2), 3) Kasumi-1 (AML M2), 4) KG-1 (AML M7), and 5) Oci-5 (AML M4), all of which are available from the ATCC. The murine leukemia cell lines of BALB/c origin were: 1) RL♂1 (radiation induced), 2) RVD, and 3) RVE (virally induced). RL♂1 is a radiation-induced leukemia in a BALB/c mouse (Nakayama E, et al., Proc. Natl. Acad. Sci USA 76:3486-3490 (1979). RVD and RVE are leukemias induced by an injection of leukemia virus into neonatal BALB/c mice (see e.g., Stockert, E, et al., J. Exp. Med. 149:200-215, 1979 and Manki, A, et al., Cancer Res 58(9):1960-1964, 1988). Murine cell lines were generously contributed by E. Nakayama, Japan.

Patient Samples

All samples were taken from patients treated in the framework of studies for AML, CML, renal cell cancer, ovarian carcinoma, breast carcinoma, and melanoma approved by the local ethics committee. Informed consent was obtained from all patients concerning the use of all their material for scientific purposes.

Peripheral blood samples anticoagulated by EDTA of untreated AML patients (the percentage of blasts of the white blood cells was 60% to 90%, median 80%) and CML patients (leukocytes>20 g/l) were collected at the time of diagnosis. Peripheral blood mononuclear cells (PBMN) were prepared by Ficoll separation using standard procedures, and stored for RNA-preparation at −80° C. Samples of tumor tissue and of tissue macroscopically free of tumor were collected from patients with solid tumors at the time of surgery. Sera of leukemia patients and patients with solid tumors were stored at −80° C. The sera were preabsorbed and diluted 1:100 as described (Greiner J, et al., Exp J Hematol 28:1413-1422, 2000).

Construction and Screening of a CML K562 cDNA Library

The phenol/chloroform method was used for total RNA preparation (Chomczynski P, Sacchi N. Anal Biochem 162:156-159, 1987). mRNA was isolated and purified with an Poly(A)+ isolation kit (mRNA Purification Kit, Pharmacia Biotech, Piscataway, N.J.). 5 μg mRNA was used for cDNA-library construction as described (Greiner J, et al., Exp J Hematol 28:1413-1422, 2000). Briefly, a representational CML K562 library was created in a ZAP lambda bacteriophage expression vector(e.g., Zap vector, Stratagene, La Jolla, Calif.), expressed in E. coli and blotted on nitrocellulose membrane with allogeneic sera preabsorbed as described (Greiner J, et al., Exp J Hematol 28:1413-1422, 2000). Positive clones were subjected to single-clone excision and plasmid extraction followed by sequencing using an ABI PRISM 310 capillary genetic analyser (Perkin Elmer, Foster City, Calif.). Sequence alignments were performed using the software and searching programs provided by the National Center of Biotechnology Information (NCBI, http://www.ncbi.nlm.nih.gov). The RHAMM clones were compared and deposited to the SEREX database (http://www.licr.org/SEREX.htm).

Conventional RT-PCR

mRNA was prepared from PBMN or tumor samples by using mRNA QuickPrep Micro purification kit (Amersham Pharmacia Biotech, Little Chalfont, England, UK). 2.0 μg of each mRNA sample was subjected to cDNA synthesis (Superscript II Gibco BRL, Frederick, Md.). PCR was performed for the three isoforms of RHAMM published (Crainie M, et al., Blood 93:1684-1696, 1999): the full length sequence of RHAMM (RHAMM^{FL}, SEQ ID NO: 1) and the splice variants RHAMM⁻⁴⁸, SEQ ID NO: 2) and RHAMM⁻¹⁴⁷, SEQ ID NO: 3). The primers for RHAMM PCR 1 were used to detect the mRNA expression of the gene RHAMM^{FL}. The primer pair for PCR 2 was used for the detection of the RHAMM⁻⁴⁸ isoform, and PCR 3 for the RHAMM⁻¹⁴⁷ variant. Patients and cell lines of hematological malignancies were screened for mRNA expression of these three isoforms of RHAMM. Furthermore, the mRNA expression was evaluated in different normal tissues with a standardized cDNA tissue panel (Multiple tissue cDNA panels, BK1420-1 and BK1421-1, Clontech, Palo Alto, Calif., USA). For standardization, the expression of the antigens was correlated with the expression level of the housekeeping gene β-actin (conventional RT-PCR) and for real-time RT-PCR the housekeeping gene TATA-box binding protein (TBP) using the following procedures:

1) RHAMM PCR 1

(SEQ ID NO:4)

| | |
|---|---|
| Forward primer: | 5′ caggtcacccaaaggagtctcg 3′ |

(SEQ ID NO:5)

| | |
|---|---|
| Reverse primer: | 5′ caagctcatccagtgtttgc 3′ |

Amplification Conditions

95° C. denaturation (1 min)

60° C. annealing (1 min)

72° C. elongation (1 min)

36 cycles, MgCl₂ 1.5 mM.

2) RHAMM PCR 2

(SEQ ID NO:6)

| | |
|---|---|
| Forward primer: | 5′ ggccgtcaacaagtcctttccta 3′ |

(SEQ ID NO:7)

| | |
|---|---|
| Reverse primer: | 5′ ttgggctattttcccttgagactc 3′ |

Amplification Conditions

95° C. denaturation (1 min)

60° C. annealing (1 min)

72° C. elongation (1 min)

35 cycles, MgCl₂ 1.5 mM

3) RHAMM PCR 3

(SEQ ID NO:8)

| | |
|---|---|
| Forward primer: | 5′ aggaggaacaagctgaaagg 3′ |

(SEQ ID NO:9)

| | |
|---|---|
| Reverse primer: | 5′ ttcctgagctgcaccatgtt 3′ |

Amplification Conditions

95° C. denaturation (1 min)

58° C. annealing (1 min)

72° C. elongation (1 min)

35 cycles, MgCl₂ 1.5 mM.

4) β-Actin

(SEQ ID NO:10)

| | |
|---|---|
| Forward primer: | 5′ gcatcgtgatggactccg 3′ |

(SEQ ID NO:11)

| | |
|---|---|
| Reverse primer: | 5′ gctggaaggtggacagcga 3′ |

Amplification Conditions

95° C. denaturation (1 min)

68° C. annealing (1 min)

72° C. elongation (1 min)

24 cycles, MgCl₂ 1.5 mM.

5) TBP

(SEQ ID NO:12)

| | |
|---|---|
| Forward primer: | 5′ cacgaaceacggcactgatt 3′ |

(SEQ ID NO:13)

| | |
|---|---|
| Reverse primer: | 5′ ttttcttgctgccagtctggac 3′. |

Amplification Conditions

95° C. denaturation (1 min)

60° C. annealing (1 min)

72° C. elongation (1 min)

40 cycles, MgCl₂ 1.5 mM.

Real-time RT-PCR

For the quantitative measurement of the mRNA expression of RHAMM in PBMN of AML patients, real-time RT-PCR using the light cycler SYBR Green I technology was performed according to the manufacturer's protocol (e.g. Molecular Probes, Eugene, Oreg.) and using the primers described above as follows: initial denaturation at 95° C. for 10 min, then 40 cycles with: 5 s at 95° C. 5 s at 64° C. 30 s at 72° C. To quantify the mRNA expression of RHAMM, the copy number of the samples was correlated with the mRNA expression of the housekeeping gene TBP (no retro-pseudogenes known) (Bieche I, et al., Cancer Res 59:2759-2765, 1999). RT-PCR for TBP was performed using the primers described above under the following conditions: initial denaturation at 95° C. for 10 min, then 40 cycles with 15 s at 95° C., 10 s at 60° C., 20 s at 72° C.

Western Blot Analysis

5×10⁶ to 1×10⁷ cells were lysed, and equal amounts of protein (50 μg/lane) were separated for sodium dodecyl sulfate-polyacrylamide gel electrophoresis (SDS-PAGE) as described (Greiner J, et al., Exp J Hematol 28:1413-1422, 2000). Separated proteins were blotted onto nitrocellulose membranes. Blots were incubated with the primary anti-RHAMM

Amplification Conditions

95° C. denaturation (1 min)

68° C. annealing (1 min)

72° C. elongation (1 min)

24 cycles, MgCl₂ 1.5 mM.

5) TBP

(SEQ ID NO:12)

| | |
|---|---|
| Forward primer: | 5′ cacgaaccacggcactgatt 3′ |

(SEQ ID NO:13)

| | |
|---|---|
| Reverse primer: | 5′ ttttcttgctgccagtctggac 3′. |

Amplification Conditions

95° C. denaturation (1 min)

60° C. annealing (1 min)

72° C. elongation (1 min)

40 cycles, MgCl₂ 1.5 mM.

Real-time RT-PCR

For the quantitative measurement of the mRNA expression of RHAMM in PBMN of AML patients, real-time RT-PCR using the light cycler SYBR Green I technology was performed according to the manufacturer's protocol (e.g. Molecular Probes, Eugene, Oreg.) and using the primers described above as follows: initial denaturation at 95° C. for 10 min, then 40 cycles with: 5 s at 95° C., 5 s at 64° C., 30 s at 72° C. To quantify the mRNA expression of RHAMM, the copy number of the samples was correlated with the mRNA expression of the housekeeping gene TBP (no retro-pseudogenes known) (Bieche I, et al., Cancer Res 59:2759-2765, 1999). RT-PCR for TBP was performed using the primers described above under the following conditions: initial denaturation at 95° C. for 10 min, then 40 cycles with 15 s at 95° C., 10 s at 60° C., 20 s at 72° C.

Western Blot Analysis

5×10⁶ to 1×10⁷ cells were lysed, and equal amounts of protein (50 μg/lane) were separated for sodium dodecyl sulfate-polyacrylamide gel electrophoresis (SDS-PAGE) as described (Greiner J, et al., Exp J Hematol 28:1413-1422, 2000). Separated proteins were blotted onto nitrocellulose membranes. Blots were incubated with the primary anti-RHAMM antibody, a rabbit polyclonal antibody (Hofmann, M, et al., J Cell Sci 111:1673-1684, 1998) specific for the human 85-90 kDa RHAMM protein. Blots were incubated in the antibody overnight at 4° C., washed three times, and incubated with peroxidase-conjugated goat anti-rabbit-IgG (1:500 dilution), and visualized using the chemoluminescence (ECL; Amersham, Quebec, Canada) technique according to the manufacturer's instructions. In the murine system, western blot analysis was performed with a polyclonal rabbit antibody detecting the murine 92-95 kDa protein of RHAMM (Hofmann, M, et al., J Cell Sci 111:1673-1684, 1998).

Results

Serological Screening of the CML cDNA Library

The expression library constructed from K562 yielded about 250,000 primary clones. The amplified library contained>1×10⁸ clones, of whom 210,000 clones were subjected to serological screening. The first screening used a mixture of four sera from CML patients. Five clones (UL-CML-1 to 5) containing the same inserts with homology to the receptor of hyaluronic acid mediated motility (RHAMM) showed positive serological reactions. The clones UL-CML-1 and UL-CML-2 were homologous from the position 214 of RHAMM to the polyA-sequence, UL-CML-3, UL-CML-4 and the clone UL-CML-5 from position 48 of RHAMM to the polyA-sequence of RHAMM. The coding sequence of RHAMM ranges from position 37 to 2166 (Wang C, et al., Gene 174:299-306, 1996).

RHAMM Serology

The clones containing the gene RHAMM were tested with 19 sera of AML patients and 16 sera of CML patients, furthermore with sera of patients with solid tumors or with autoimmune diseases and with sera from healthy volunteers. A summary of the data from the serological screening is shown in Table 1.

Positive serological reactions to the clones containing RHAMM were found in patients with different tumor entities (AML, CML, renal cell cancer, melanoma metastases, breast cancer, ovarian cancer). No serological reactions to the clones containing RHAMM were found in healthy volunteers and patients with autoimmune diseases.

TABLE 1

| | |
|---|---|
| RHAMM | Positive serology |

| | | |
|---|---|---|
| AML | 8/19 | 42% |
| CML | 5/16 | 31% |
| RCC | 4/10 | 40% |
| Met. melanoma | 5/6 | 83% |
| Breast carcinoma | 8/12 | 67% |
| Ovarian carcinoma | 5/10 | 50% |
| Healthy volunteers | 0/12 | 0% |
| Autoimmune diseases | 0/7 | 0% |

mRNA Expression of RHAMM

High mRNA expression was found in the cell line K562, serving as a positive control and in the other leukemia cell lines HL-60, Kasumi-1, KG-1, and Oci-5. Of the AML patients, 14/40 (35%) showed high, 10/40 (25%) lower, and 16/40 (40%) no mRNA expression of the antigen RHAMM. Of the CML patients, 2/15 (13%) showed high, 4/15 (27%) lower, and 9/15 (60%) no mRNA expression of RHAMM. No RHAMM mRNA expression was detectable in PBMN and CD34 positive separated cell samples of healthy volunteers. Fifteen samples of RCC patients were examined for mRNA expression of RHAMM and 4/15 (27%) of the RCC tumor samples showed high, 7/15 (47%) lower, and 4/15 (27%) no mRNA expression of RHAMM. All samples (10/10) of normal renal tissue samples showed no RHAMM mRNA expression. RHAMM mRNA expression was also found in 4/5 (80%) of melanoma metastases, but not in primary melanoma, naevi, or normal skin. RHAMM mRNA expression also was found in 3/5 (60%) of breast carcinomas and in 5/10 (50%) of ovarian carcinomas. RHAMM mRNA expression was not detected in peripheral blood mononuclear cell samples of healthy volunteers, CD34-positive selected samples from patients without hematological malignancies or healthy donors. A summary of the data is shown in Table 2.

The mRNA expression of the three isoforms RHAMM^{FL}, RHAMM⁻⁴⁸, and RHAMM⁻¹⁴⁷ was examined in samples of AML, CML, and RCC patients, and human and murine cell lines of hematological malignancies. RHAMM PCR 1 was used for detection of the full-length gene of RHAMM, the PCR 2 and 3 for the RHAMM⁻⁴⁸ and RHAMM⁻¹⁴⁷ splice variants respectively. The splice variant RHAMM⁻⁴⁸ was detected in 24/24 (100%) of AML, 5/6 (83%) of CML and 11/11 RCC patients (100%) with positive mRNA expression of the full-length isoform of RHAMM. None of the patient samples showed expression of the isoform RHAMM⁻¹⁴⁷. In all human leukemia cell lines expression of the RHAMM⁻⁴⁸ splice variant was detectable, whereas the RHAMM⁻¹⁴⁷ isoform was not found.

TABLE 2

| | |
|---|---|
| RHAMM | mRNA expression |

| | | |
|---|---|---|
| AML | 24/40 | 60% |
| CML | 6/15 | 40% |
| RCC | 11/15 | 73% |
| Met. Melanoma | 4/5 | 80% |
| Prim. Melanoma | 0/5 | 0% |
| Breast carcinoma | 3/5 | 60% |
| Ovarian carcinoma | 5/10 | 50% |
| Naevi | 0/5 | 0% |
| Normal skin | 0/5 | 0% |
| PBMN of HV | 0/12 | 0% |
| CD 34 cells of HV | 0/10 | 0% |
| Normal kidney | 0/10 | 0% |

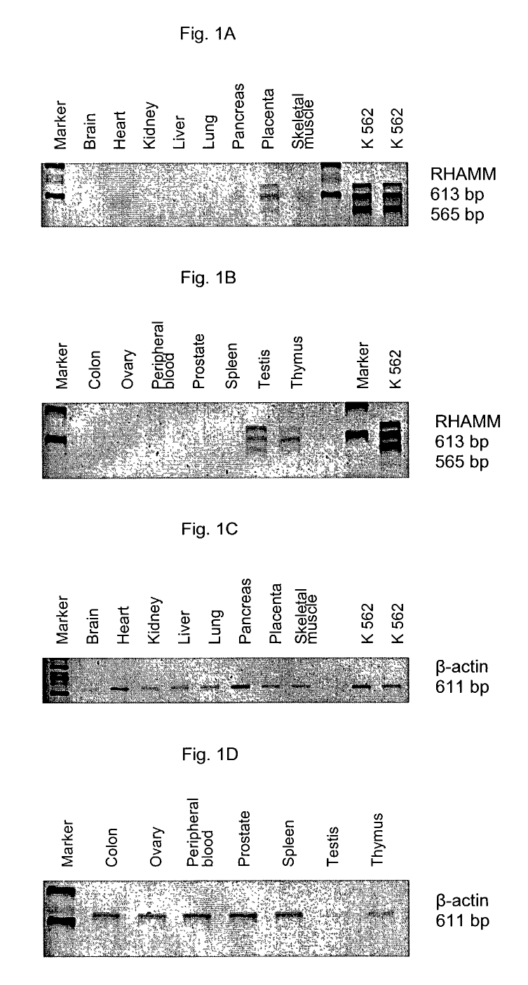

In normal tissues, high mRNA expression of RHAMM was only detectable in testis, thymus, and placenta. Very low mRNA expression was shown in normal pancreas and lung tissue. No mRNA expression was found in all other normal tissues (FIGS. 1A-D).

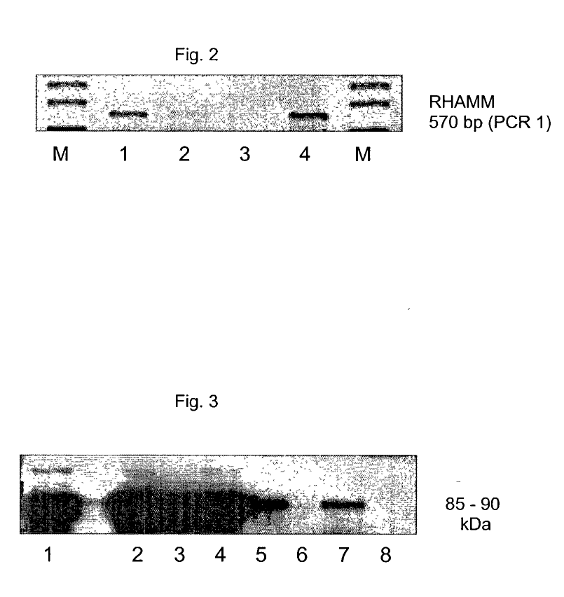

Two AML patients were examined for their mRNA expression of RHAMM during induction polychemotherapy resulting in a complete remission in peripheral blood and bone marrow. Before treatment, high mRNA expression of RHAMM was detectable in peripheral blood samples. After treatment of these patients with two courses of chemotherapy, consisting of idarubicine 12 mg/m² d 3-5, arabinoside C 100 mg/m² d 1-6, and etoposide 100 mg/m² d 2-6, no mRNA expression of RHAMM was detectable. FIG. 2 shows the decrease of RHAMM mRNA expression during the course of induction polychemotherapy of an AML patient.

Quantitative mRNA Expression of RHAMM Using Real-time RT-PCR

RHAMM cDNA was isolated with conventional PCR procedures the cDNA was measured by photometry. A defined copy number of cDNA was used for preparation of standard curves. For quantification of the antigen RHAMM, the mRNA expression of RHAMM was correlated with the mRNA expression of the housekeeping gene TBP (TATA-box binding protein). AML patients, leukemia cell lines. and PBMN of healthy volunteers were examined with real-time RHAMM PCR. Control reactions with 0.00125 ng, 0.0125 ng, 0.125 ng, 1.25 ng and 12.5 ng RHAMM were also run. No mRNA expression of RHAMM was detectable in PBMN of healthy volunteers, whereas high expression was found in all leukemia cell lines and in these AML patients. In the cell line K562 high mRNA expression of RHAMM was detectable. 50 ng cDNA of the leukemia cell line K562 yielded a starting amount of RHAMM of 5 pg. Two AML patients show high and lower mRNA expression of RHAMM. No significant RHAMM mRNA expression was found in PBMN of healthy volunteers, the amplification curves showed no exponential growth. mRNA expression levels evaluated by real-time RT-PCR correlated with mRNA expression levels of conventional RT-PCR.

Western Blot Analysis

Western blot analysis of RHAMM was performed in leukemia cell lines, PBMN of AML patients, and healthy volunteers. The cell line K562 showed high protein expression of RHAMM and was used as a positive control. High protein expression was also detected in the other human leukemia cell lines HL-60, Kasumi-1, KG-1, and Oci-5 (5/5). RHAMM protein expression was also detected in murine leukemia cell lines. Protein expression was found in 7/10 (70%) PBMN of AML patients. In all PBMN samples of healthy volunteers (0/10) no protein expression of the antigen RHAMM was found (FIG. 3 shows representative results).

TABLE 3
Nucleotide and amino acid correspondence

| Nucleotide SEQ ID NO. | Amino Acid SEQ ID NO. |
|---|---|
| SEQ ID NO: 1 | SEQ ID NO: 14 |
| SEQ ID NO: 2 | SEQ ID NO: 15 |
| SEQ ID NO: 3 | SEQ ID NO: 16 |

Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

We claim:

1. A method for diagnosing cancer in a subject comprising:
obtaining a biological sample from a subject,
contacting the sample with a RHAMM polypeptide encoded by a nucleic acid molecule comprising a nucleotide sequence selected from the group consisting of SEQ ID NO: 1, 2, and 3, and
determining specific binding between the RHAMM polypeptide and agents in the sample, wherein the presence of specific binding is diagnostic for cancer in the subject.

2. The method of claim 1, wherein the sample is a blood sample.

3. The method of claim 1, wherein the agents are antibodies or antigen-binding fragments thereof.

4. The method of claim 1, wherein the cancer is acute or chronic myeloid leukemia.

5. The method of claim 1, wherein the cancer is selected from the group consisting of: renal cell cancer, metastatic melanoma, and ovarian carcinoma.

6. A method for determining onset, progression, or regression, of cancer in a subject, comprising:
obtaining from a subject at a first time a first biological sample,
determining specific binding between agents in the first sample and a RHAMM polypeptide encoded by a nucleic acid molecule comprising a nucleotide sequence selected from the group consisting of SEQ ID NO: 1, 2, and 3, and
obtaining from a subject at a subsequent time a second biological sample,
determining specific binding between agents in the second sample and a RHAMM polypeptide encoded by a nucleic acid molecule comprising a nucleotide sequence selected from the group consisting of SEQ ID NO: 1, 2, and 3, and
comparing the determination of binding in the first sample to the determination of specific binding in the second sample as a determination of the onset, progression, or regression of the cancer.

7. The method of claim 6, wherein the sample is a blood sample.

8. The method of claim 6, wherein the agents are antibodies or antigen-binding fragments thereof.

9. The method of claim 6, wherein the cancer is acute or chronic myeloid leukemia.

10. The method of claim 6, wherein the cancer is selected from the group consisting of: renal cell cancer, metastatic melanoma, and ovarian carcinoma.

11. A method for selecting a course of treatment of a subject having or suspected of having cancer, comprising:
obtaining from the subject a biological sample,
contacting the sample with a RHAMM polypeptide encoded by a nucleic acid molecule comprising a nucleotide sequence selected from the group consisting of SEQ ID NO: 1, 2, and 3, and
determining specific binding between agents in the sample that are differentially expressed in different types of cancer, and the RHAMM polypeptide, and
selecting a course of treatment appropriate to the cancer of the subject.

12. The method of claim 11, wherein the treatment is administering antibodies that specifically bind to the RHAMM polypeptide.

13. The method of claim 12, wherein the antibodies are labeled with one or more cytotoxic agents.

14. The method of claim 11, wherein the sample is a blood sample.

15. The method of claim 11, wherein the agents are antibodies or antigen-binding fragments thereof.

16. The method of claim 11, wherein the cancer is acute or chronic myeloid leukemia.

17. The method of claim 11, wherein the cancer is selected from the group consisting of: renal cell cancer, metastatic melanoma, and ovarian carcinoma.

18. A kit for the diagnosis of cancer in a subject, comprising:
a RHAMM polypeptide encoded by a nucleic acid molecule comprising a nucleotide sequence selected from the group consisting of SEQ ID NO: 1, 2, and 3, and one or more control antigens; and instructions for the use of the polypeptide and control antigens in the diagnosis of cancer.

19. The kit of claim 18, wherein the RHAMM polypeptide is bound to a substrate.

20. The kit of claim 18, wherein the one or more agents are antibodies or antigen-binding fragments thereof.

21. The kit of claim 18, wherein the cancer is acute or chronic myeloid leukemia.

22. The kit of claim 18, wherein the cancer is selected from the group consisting of: renal cell cancer, metastatic melanoma, and ovarian carcinoma.

23. A protein microarray comprising of a RHAMM polypeptide, wherein the RHAMM polypeptide is encoded by a nucleic acid molecule comprising a nucleotide sequence selected from the group consisting of SEQ ID NO: 1, 2, and 3, and fixed to a solid substrate.

24. The protein microarray of claim 23, further comprising at least one control polypeptide molecule.
